(19) United States
(12) Patent Application Publication
(10) Pub. No.: US 2026/0124614 A1
(43) Pub. Date: May 7, 2026

(54) BIOSENSOR AND METHOD OF MANUFACTURING THE SAME

(71) Applicant: Leap BioChip Co., Ltd., Hsinchu City (TW)

(72) Inventor: Chiun-Jye Yuan, Hsinzhu County (TW)

(21) Appl. No.: 18/988,914

(22) Filed: Dec. 20, 2024

(30) Foreign Application Priority Data
Nov. 1, 2024 (TW) 113141809

(51) Int. Cl. B01L 3/00; G01N 33/543

(52) U.S. Cl. CPC B01L 3/502715; B01L 3/502707; B01L 2300/0645; B01L 2300/0883; B01L 2300/16; G01N 33/5438

(57) ABSTRACT

A biosensor and a biosensor manufacturing method. The biosensor includes a carrier substrate, a first insulating layer, a second insulating layer, a first conductive circuit layer, a second conductive circuit layer, multiple first conductive through layers, multiple second conductive through layers, multiple first exposed electrodes and multiple second exposed electrodes. Each first exposed electrode is electrically connected to the first conductive through layer. Each second exposed electrode is electrically connected to the second conductive through layer. The first exposed electrode can be electrically connected to the first conductive circuit layer through the corresponding first conductive through layer and be separate from the first conductive circuit layer by a first predetermined distance. The second exposed electrode can be electrically connected to the second conductive circuit layer through the corresponding second conductive through layer and be separate from the second conductive circuit layer by a second predetermined distance.

### CROSS-REFERENCE TO RELATED PATENT APPLICATION

This application claims the benefit of priority to Taiwan Patent Application No. 113141809, filed on Nov. 1, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a sensor and a method of manufacturing the same, and more particularly to a biosensor and a method of manufacturing the same.

### BACKGROUND OF THE DISCLOSURE

Biosensors can return responses of biomolecules (such as enzymes, antibodies, cell receptors or DNA probes) through physical or chemical detectors, and then present the sensing results to the user through a signal processor. However, the biosensors in the related art still have room for improvement.

### SUMMARY OF THE DISCLOSURE

In response to the above-referenced technical inadequacy, the present disclosure provides a biosensor and a method of manufacturing the same.

In order to solve the above-mentioned problems, one of the technical aspects adopted by the present disclosure is to provide a biosensor, which includes a carrier substrate, a first insulating layer, a first conductive circuit layer, a second conductive circuit layer, a second insulating layer, a plurality of first conductive through layers, a plurality of second conductive through layers, a plurality of first exposed electrodes and a plurality of second exposed electrodes. The first insulating layer is disposed on the carrier substrate. The first conductive circuit layer is disposed on the first insulating layer. The second conductive circuit layer is disposed on the first insulating layer. The second insulating layer is disposed on the first insulating layer for partially covering the first conductive circuit layer and the second conductive circuit layer. The plurality of first conductive through layers pass through the second insulating layer to be electrically connected to the first conductive circuit layer. The plurality of second conductive through layers pass through the second insulating layer to be electrically connected to the second conductive circuit layer. The plurality of first exposed electrodes are disposed on the second insulating layer, and each of the plurality of first exposed electrodes is electrically connected to a corresponding one of the plurality of first conductive through layers. The plurality of second exposed electrodes are disposed on the second insulating layer, and each of the plurality of second exposed electrodes is electrically connected to a corresponding one of the plurality of second conductive through layers. The first conductive circuit layer and the second conductive circuit layer are adjacent to each other and separate from each other, and the first conductive circuit layer and the second conductive circuit layer cooperate with each other to form an interdigitated electrode structure. The first conductive circuit layer includes a first extension portion and a plurality of first circuit portions extending from the first extension portion, the second conductive circuit layer includes a second extension portion and a plurality of second circuit portions extending from the second extension portion, and the plurality of first circuit portions of the first conductive circuit layer and the plurality of second circuit portions of the second conductive circuit layer are alternately arranged.

In order to solve the above-mentioned problems, another one of the technical aspects adopted by the present disclosure is to provide a biosensor, which includes a carrier substrate, a first insulating layer, a first conductive circuit layer, a second conductive circuit layer, a second insulating layer, a plurality of first conductive through layers, a plurality of second conductive through layers, a plurality of first exposed electrodes and a plurality of second exposed electrodes. The first insulating layer is disposed on the carrier substrate. The first conductive circuit layer is disposed on the first insulating layer. The second conductive circuit layer is disposed on the first insulating layer. The second insulating layer is disposed on the first insulating layer for partially covering the first conductive circuit layer and the second conductive circuit layer. The plurality of first conductive through layers pass through the second insulating layer to be electrically connected to the first conductive circuit layer. The plurality of second conductive through layers pass through the second insulating layer to be electrically connected to the second conductive circuit layer. The plurality of first exposed electrodes are disposed on the second insulating layer, and each of the plurality of first exposed electrodes is electrically connected to a corresponding one of the plurality of first conductive through layers. The plurality of second exposed electrodes are disposed on the second insulating layer, and each of the plurality of second exposed electrodes is electrically connected to a corresponding one of the plurality of second conductive through layers.

In order to solve the above-mentioned problems, yet another one of the technical aspects adopted by the present disclosure is to provide a biosensor manufacturing method, which includes: forming a first insulating layer disposed on a carrier substrate; forming a first conductive circuit layer and a second conductive circuit layer on the first insulating layer; forming a second insulating layer on the first insulating layer for partially covering the first conductive circuit layer and the second conductive circuit layer; forming a plurality of first conductive through layers and a plurality of second conductive through layers, in which the plurality of first conductive through layers pass through the second insulating layer to be electrically connected to the first conductive circuit layer, and the plurality of second conductive through layers pass through the second insulating layer to be electrically connected to the second conductive circuit layer; and forming a plurality of first exposed electrodes and a plurality of second exposed electrodes, in which the plurality of first exposed electrodes are disposed on the second insulating layer and electrically connected to the plurality of first conductive through layers, respectively, and the plurality of second exposed electrodes are disposed on the second insulating layer and electrically connected to the plurality of second conductive through layers, respectively.

Therefore, in the biosensor provided by the present disclosure, by virtue of “the plurality of first conductive through layers passing through the second insulating layer to be electrically connected to the first conductive circuit layer,” “the plurality of second conductive through layers passing through the second insulating layer to be electrically connected to the second conductive circuit layer,” “the plurality of first exposed electrodes being disposed on the second insulating layer, and each of the plurality of first exposed electrodes being electrically connected to a corresponding one of the plurality of first conductive through layers” and “the plurality of second exposed electrodes being disposed on the second insulating layer, and each of the plurality of second exposed electrodes being electrically connected to a corresponding one of the plurality of second conductive through layers,” the first exposed electrode can be electrically connected to the first conductive circuit layer through the corresponding first conductive through layer and be separate from the first conductive circuit layer by a first predetermined distance, and the second exposed electrode can be electrically connected to the second conductive circuit layer through the corresponding second conductive through layer and be separate from the second conductive circuit layer by a second predetermined distance.

Furthermore, in the biosensor manufacturing method provided by the present disclosure, by virtue of “forming a plurality of first conductive through layers and a plurality of second conductive through layers, the plurality of first conductive through layers passing through the second insulating layer to be electrically connected to the first conductive circuit layer, and the plurality of second conductive through layers passing through the second insulating layer to be electrically connected to the second conductive circuit layer” and “forming a plurality of first exposed electrodes and a plurality of second exposed electrodes, the plurality of first exposed electrodes being disposed on the second insulating layer and electrically connected to the plurality of first conductive through layers, respectively, and the plurality of second exposed electrodes being disposed on the second insulating layer and electrically connected to the plurality of second conductive through layers, respectively,” the first exposed electrode can be electrically connected to the first conductive circuit layer through the corresponding first conductive through layer and be separate from the first conductive circuit layer by a first predetermined distance, and the second exposed electrode can be electrically connected to the second conductive circuit layer through the corresponding second conductive through layer and be separate from the second conductive circuit layer by a second predetermined distance.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a biosensor manufacturing method according to a first embodiment of the present disclosure;

FIG. 2 is a schematic top view of step S102 of the biosensor manufacturing method according to the first embodiment of the present disclosure;

FIG. 3 is a partial cross-sectional view taken along line III-III in FIG. 2;

FIG. 4 is a schematic top view of step S104 of the biosensor manufacturing method according to the first embodiment of the present disclosure;

FIG. 5 is a partial cross-sectional view taken along line V-V in FIG. 4;

FIG. 6 is a schematic top view of step S106 of the biosensor manufacturing method according to the first embodiment of the present disclosure;

FIG. 7 is a partial cross-sectional view taken along line VII-VII in FIG. 6;

FIG. 8 is a schematic top view of step S108 of the biosensor manufacturing method according to the first embodiment of the present disclosure (or a schematic top view of a biosensor according to the first embodiment of the present disclosure);

FIG. 9 is a partial cross-sectional view taken along line IX-IX in FIG. 8; and

FIG. 10 is a schematic top view of the biosensor according to a second embodiment of the present disclosure.

### DETAILED DESCRIPTION OF THE EXEMPLARY EMBODIMENTS

The present disclosure is more particularly described in the following embodiments and examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

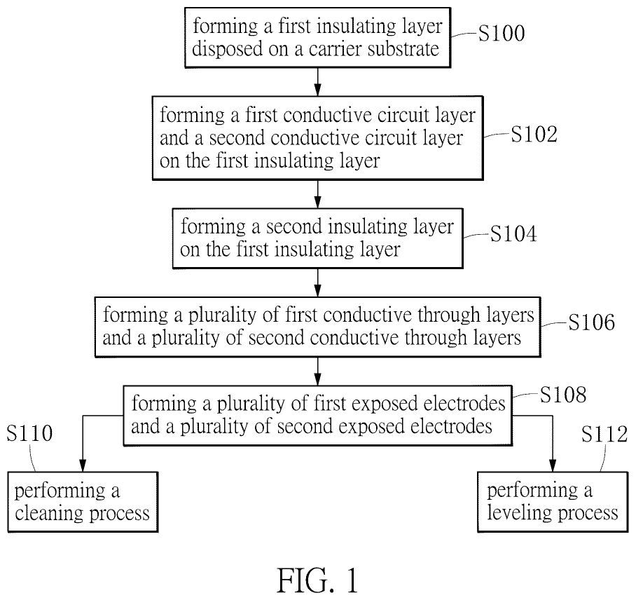

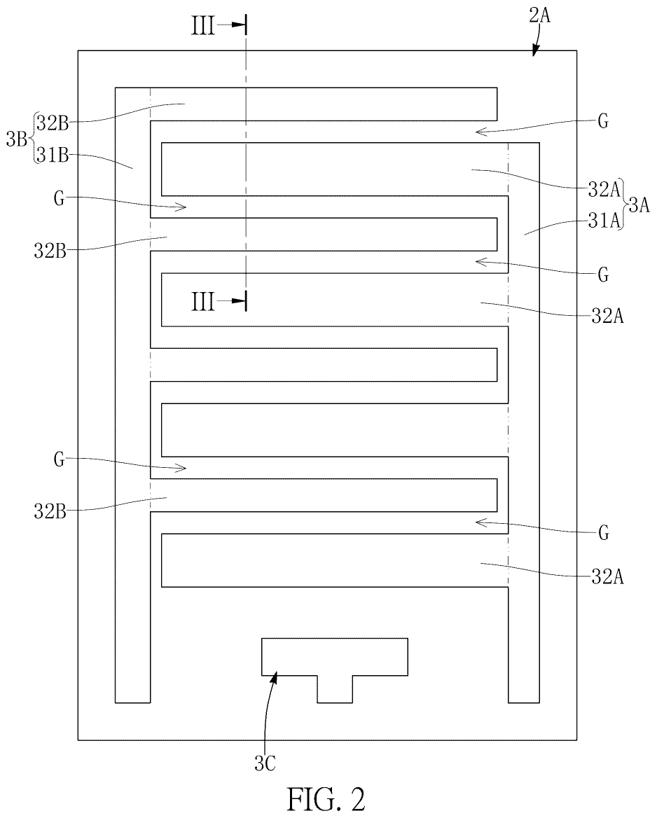

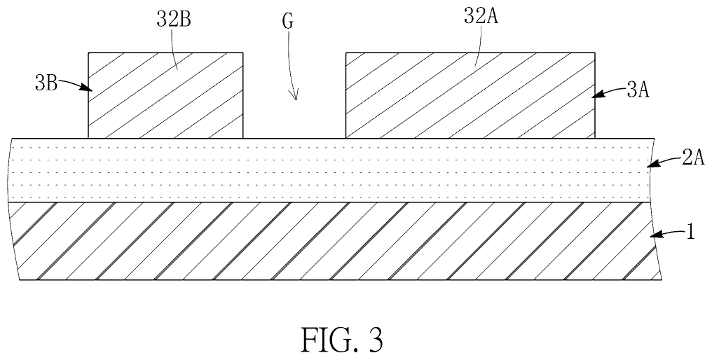

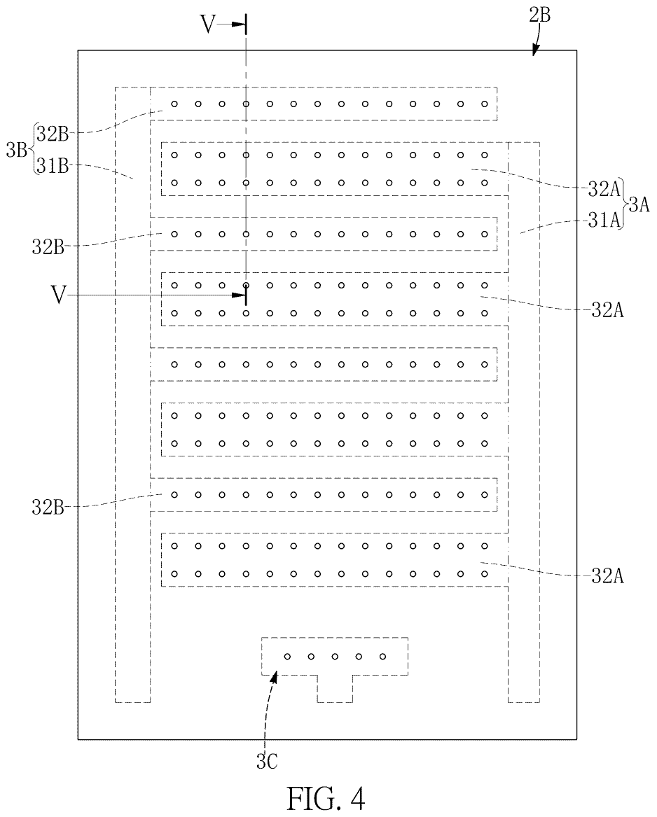

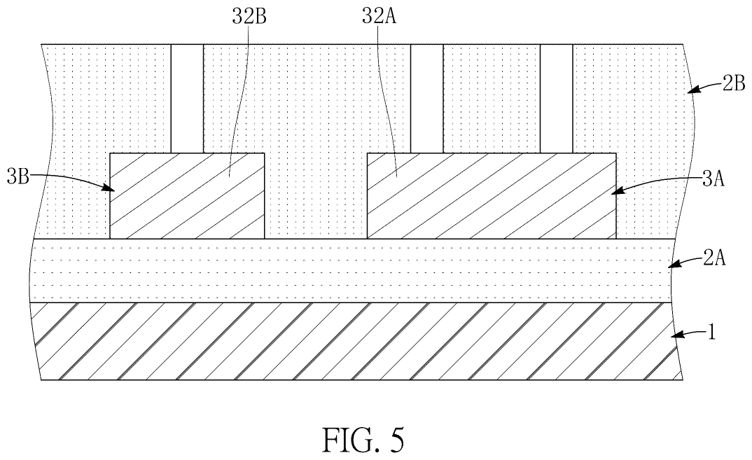

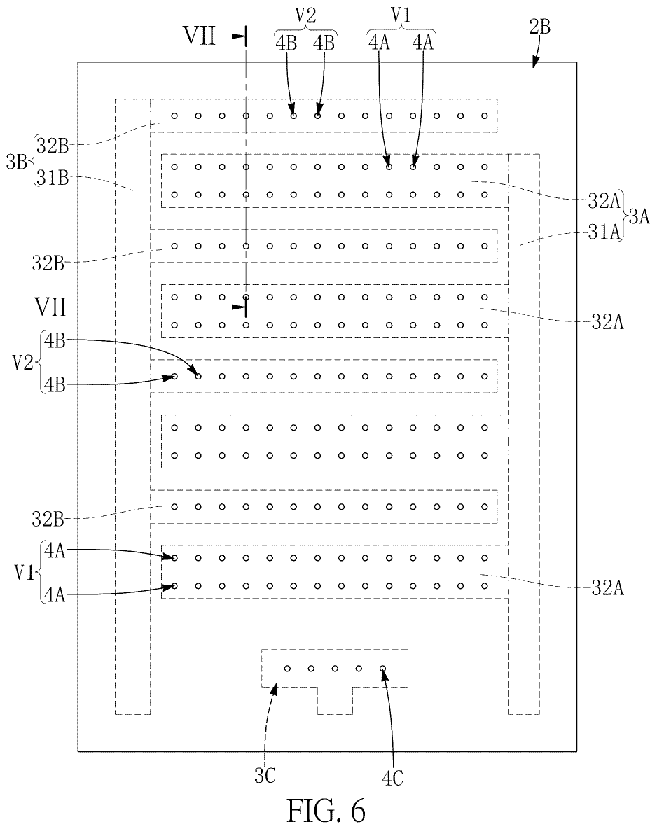

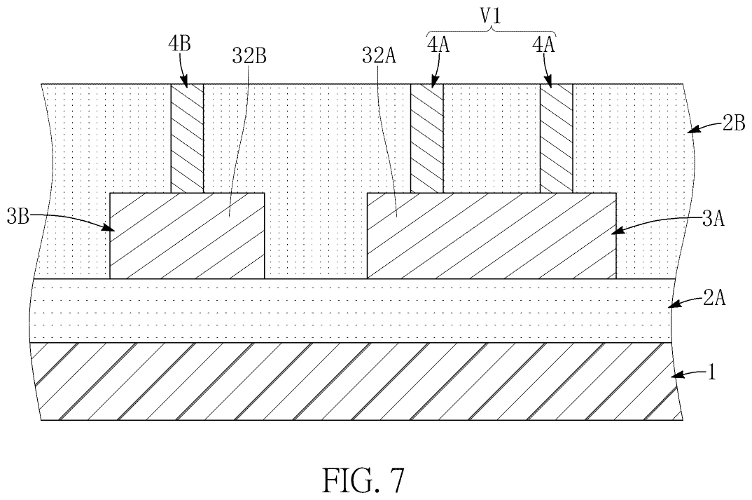

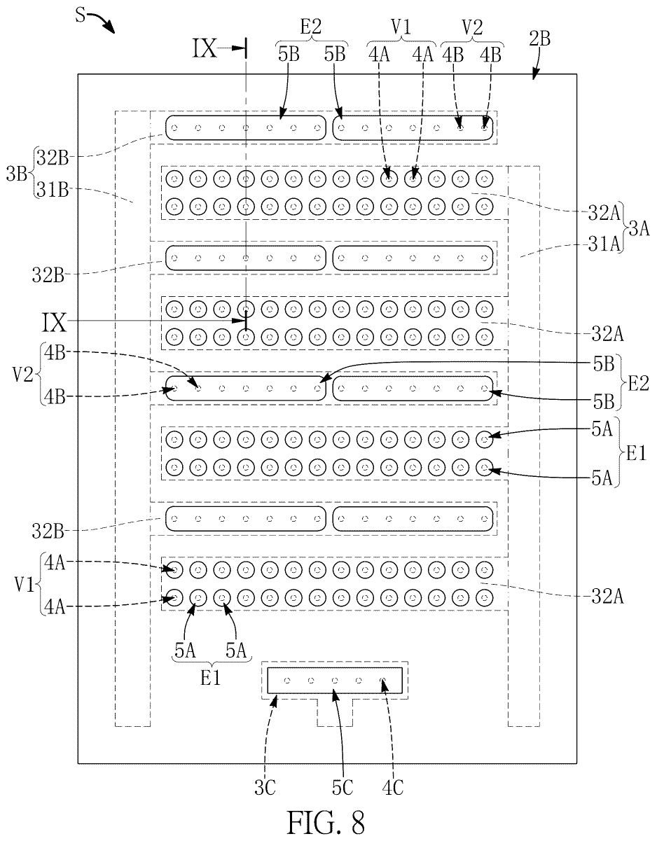

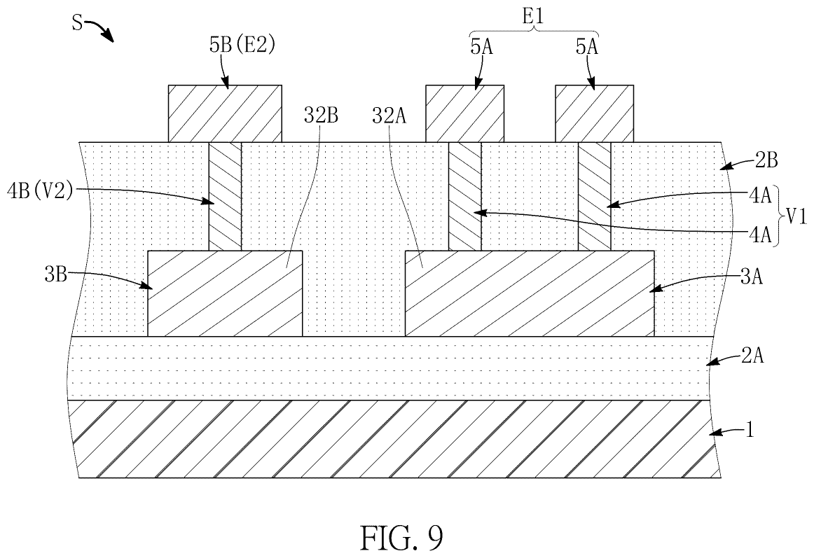

Referring to FIG. 1 to FIG. 9, a first embodiment of the present disclosure provides a biosensor manufacturing method, which may include at least the following processes or steps: firstly, referring to FIG. 1, FIG. 2 and FIG. 3, forming a first insulating layer 2A disposed on a carrier substrate 1 (step S100), and then forming a first conductive circuit layer 3A (or a first conductive layout layer) and a second conductive circuit layer 3B (or a second conductive layout layer) on the first insulating layer 2A (step S102); next, referring to FIG. 1, FIG. 4 and FIG. 5, forming a second insulating layer 2B on the first insulating layer 2A for partially covering the first conductive circuit layer 3A and the second conductive circuit layer 3B (step S104); then, referring to FIG. 1, FIG. 6 and FIG. 7, forming a plurality of first conductive through layers 4A (or first conductive penetration layers) and a plurality of second conductive through layers 4B (or second conductive penetration layers), in which the first conductive through layers 4A pass through the second insulating layer 2B to be electrically connected to the first conductive circuit layer 3A, and the second conductive through layers 4B pass through the second insulating layer 2B to be electrically connected to the second conductive circuit layer 3B (step S106); and then referring to FIG. 1, FIG. 8 and FIG. 9, forming a plurality of first exposed electrodes 5A and a plurality of second exposed electrodes 5B, in which the first exposed electrodes 5A are disposed on the second insulating layer 2B and electrically connected to the first conductive through layers 4A, respectively, and the second exposed electrodes 5B are disposed on the second insulating layer 2B and electrically connected to the second conductive through layers 4B, respectively (step S108), thereby completing the production of the biosensor S.

For example, referring to FIG. 2 and FIG. 9, in the step S100 of forming the first insulating layer 2A disposed on the carrier substrate 1, the first insulating layer 2A can be completed through a semiconductor process (such as exposure, development and etching) or a non-semiconductor process. Furthermore, in the step S102 of forming the first conductive circuit layer 3A and the second conductive circuit layer 3B on the first insulating layer 2A, the first conductive circuit layer 3A and the second conductive circuit layer 3B can be completed through a semiconductor process (such as exposure, development and etching) or a non-semiconductor process. Moreover, in the step S104 of forming the second insulating layer 2B on the first insulating layer 2A, the second insulating layer 2B can be completed through a semiconductor process (such as exposure, development and etching) or a non-semiconductor process. In addition, in the step S106 of forming the first conductive through layers 4A and the second conductive through layers 4B, the first conductive through layers 4A and the second conductive through layers 4B can be completed through a semiconductor process (such as exposure, development and etching) or a non-semiconductor process. In addition, in the step S108 of forming the first exposed electrodes 5A and the second exposed electrodes 5B, the first exposed electrodes 5A and the second exposed electrodes 5B can be completed through a semiconductor process (such as exposure, development and etching) or a non-semiconductor process. However, the aforementioned details are disclosed for exemplary purposes only, and are not meant to limit the scope of the present disclosure.

It should be noted that, for example, after the process (the step S108) of forming the first exposed electrodes 5A and the second exposed electrodes 5B, the biosensor manufacturing method further includes: performing a cleaning process (step S110), in which the cleaning process can be configured as a chemical cleaning, a water cleaning, a plasma cleaning or any kind of semiconductor cleaning to clean a biosensor S manufactured by the biosensor manufacturing method provided by the present disclosure. Furthermore, after the process (the step S108) of forming the first exposed electrodes 5A and the second exposed electrodes 5B, the biosensor manufacturing method further includes: performing a leveling process (step S112), in which the leveling process (or an electrode surface leveling step) can be configured to perform surface treatment on the top surface of the first exposed electrode 5A and the top surface of the second exposed electrode 5B through physical or chemical means, thereby improving the surface flatness of each of the first exposed electrodes 5A and the surface flatness of each of the second exposed electrodes 5B. However, the aforementioned details are disclosed for exemplary purposes only, and are not meant to limit the scope of the present disclosure.

Furthermore, referring to FIG. 8 and FIG. 9, the first embodiment of the present disclosure further provides a biosensor S, which includes a carrier substrate 1, a first insulating layer 2A, a second insulating layer 2B, a first conductive circuit layer 3A, a second conductive circuit layer 3B, a plurality of first conductive through layers 4A, a plurality of second conductive through layers 4B, a plurality of first exposed electrodes 5A and a plurality of second exposed electrodes 5B. More particularly, the first insulating layer 2A is disposed on the carrier substrate 1, the first conductive circuit layer 3A is disposed on the first insulating layer 2A, and the second conductive circuit layer 3B is disposed on the first insulating layer 2A. Moreover, the second insulating layer 2B is disposed on the first insulating layer 2A for partially covering the first conductive circuit layer 3A and the second conductive circuit layer 3B, the first conductive through layers 4A pass through the second insulating layer 2B to be electrically connected to the first conductive circuit layer 3A, and the second conductive through layers 4B pass through the second insulating layer 2B to be electrically connected to the second conductive circuit layer 3B. Furthermore, the first exposed electrodes 5A are disposed on the second insulating layer 2B, and each of the first exposed electrodes 5A is electrically connected to a corresponding one of the first conductive through layers 4A. In addition, the second exposed electrodes 5B are disposed on the second insulating layer 2B, and each of the second exposed electrodes 5B is electrically connected to a corresponding one of the second conductive through layers 4B. It should be noted that, for example, as shown in FIG. 2, the first conductive circuit layer 3A and the second conductive circuit layer 3B can be adjacent to each other and separate from each other, and the first conductive circuit layer 3A and the second conductive circuit layer 3B can cooperate with each other to form an interdigitated electrode structure (or an interdigitated array electrode). More particularly, the first conductive circuit layer 3A may include a first extension portion 31A and a plurality of first circuit portions 32A extending from the first extension portion 31A, the second conductive circuit layer 3B may include a second extension portion 31B and a plurality of second circuit portions 32B extending from the second extension portion 31B, and the first circuit portions 32A of the first conductive circuit layer 3A and the second circuit portions 32B of the second conductive circuit layer 3B can be arranged alternately. However, the aforementioned details are disclosed for exemplary purposes only, and are not meant to limit the scope of the present disclosure.

For example, referring to FIG. 2 and FIG. 3, the first circuit portions 32A of the first conductive circuit layer 3A can be parallel to each other or non-parallel to each other, and the first circuit portions 32A can extend vertically or obliquely from the first extension portion 31A. In addition, the second circuit portions 32B of the second conductive circuit layer 3B can be parallel to each other or non-parallel to each other, and the second circuit portions 32B can extend vertically or obliquely from the second extension portion 31B. More particularly, the first extension portion 31A of the first conductive circuit layer 3A and the second extension portion 31B of the second conductive circuit layer 3B can be parallel to each other or non-parallel to each other, and the first circuit portions 32A of the first conductive circuit layer 3A and the second circuit portions 32B of the second conductive circuit layer 3B can be parallel to each other or non-parallel to each other. In addition, the first conductive circuit layer 3A and the second conductive circuit layer 3B can cooperate with each other to form a continuous meandering gap G (or continuous serpentine gap, or a continuous S-shaped separation space) between the first conductive circuit layer 3A and the second conductive circuit layer 3B, and the width (or the maximum width) of the continuous meandering gap G can be between 2000 nm and 6000 nm (such as any positive integer between 2000 nm and 6000 nm). However, the aforementioned details are disclosed for exemplary purposes only, and are not meant to limit the scope of the present disclosure.

For example, referring to FIG. 6 and FIG. 7, a first end and a second end of each of the first conductive through layers 4A can be in electrical contact with the first conductive circuit layer 3A and a corresponding one of the first exposed electrodes 5A, respectively (as shown in FIG. 9), and a first end and a second end of each of the second conductive through layers 4B can be in electrical contact with the second conductive circuit layer 3B and a corresponding one of the second exposed electrodes 5B, respectively (as shown in FIG. 9). It should be noted that the first conductive through layers 4A can be divided into a plurality of first via arrays V1 (or first conductive via arrays), and the first conductive through layers 4A of each of the first via arrays V1 can be electrically connected to a corresponding one of the first circuit portions 32A of the first conductive circuit layer 3A. In addition, the second conductive through layers 4B can be divided into a plurality of second via arrays V2 (or second conductive via arrays), and the second conductive through layers 4B of each of the second via arrays V2 can be electrically connected to a corresponding one of the second circuit portions 32B of the second conductive circuit layer 3B. However, the aforementioned details are disclosed for exemplary purposes only, and are not meant to limit the scope of the present disclosure.

For example, referring to FIG. 8 and FIG. 9, each of the first exposed electrodes 5A can be configured to present a columnar shape (such as a cylindrical column, a polygonal column or an arbitrary-shaped column), and each of the second exposed electrodes 5B can be configured to present a strip shape (such as a long strip or a short strip). Moreover, each of the first exposed electrodes 5A can be electrically connected to one or more corresponding ones of the first conductive through layers 4A (taking a first conductive through layer 4A as an example for description in the first embodiment), and each of the second exposed electrodes 5B can be electrically connected to one or more corresponding ones of the second conductive through layers 4B (taking a plurality of second conductive through layers 4B as an example for description in the first embodiment). In addition, the surface of each first exposed electrode 5A may not need to be provided with biological probes, or may also be provided with biological probes (such as antibodies, proteins, polypeptides, receptors, aptamers, chemical polymers, microparticles, nucleic acids or combinations thereof), and the surface of each second exposed electrode 5B may not need to be provided with biological probes, or may also be provided with biological probes (such as antibodies, proteins, polypeptides, receptors, aptamers, chemical polymers, microparticles, nucleic acids or combinations thereof). It should be noted that the first exposed electrodes 5A can be divided into a plurality of first electrode arrays E1, and the first exposed electrodes 5A of each of the first electrode arrays E1 can be disposed above a corresponding one of the first circuit portions 32A of the first conductive circuit layer 3A and electrically connected to the first conductive through layers 4A of a corresponding one of the first via arrays V1, respectively. In addition, the second exposed electrodes 5B can be divided into a plurality of second electrode arrays E2, and the second exposed electrodes 5B of each of the second electrode arrays E2 can be disposed above a corresponding one of the second circuit portions 32B of the second conductive circuit layer 3B and electrically connected to the second conductive through layers 4B of a corresponding one of the second via arrays V2, respectively. However, the aforementioned details are disclosed for exemplary purposes only, and are not meant to limit the scope of the present disclosure.

For example, referring to FIG. 2 and FIG. 3, depending on different requirements, the carrier substrate 1 can be configured as a silicon wafer substrate, a gallium nitride (GaN) substrate, a silicon carbide (SiC) substrate, a silicon germanium (SiGe) substrate, a sapphire substrate, a glass substrate or any kind of carrier substrate. In addition, depending on different requirements, the first insulating layer 2A can be configured as a first oxide layer, a first nitride layer, a first oxynitride layer or any kind of first insulating material layer, and the second insulating layer 2B can be configured as a second oxide layer, a second nitride layer, a first oxynitride layer or any kind of second insulating material layer. It should be noted that, according to different requirements, the thickness (or the height) of the first insulating layer 2A can be between 3500 Å and 4500 Å (such as any positive integer between 3500 Å and 4500 Å), and the thickness (or the height) of the second insulating layer 2B can be between 4000 Å and 15000 Å (such as any positive integer between 4000 Å and 15000 Å). However, the aforementioned details are disclosed for exemplary purposes only, and are not meant to limit the scope of the present disclosure.

For example, referring to FIG. 2 and FIG. 3, depending on different requirements, the first conductive circuit layer 3A can be configured as a first gold circuit layer made of gold (Au), a first silver circuit layer made of silver (Ag), a first copper circuit layer made of copper (Cu), a first aluminum circuit layer made of aluminum (Al), a first nickel circuit layer made of nickel (Ni), a first titanium circuit layer made of titanium (Ti), a first platinum circuit layer made of platinum (Pt), a first palladium circuit layer made of palladium (Pd), a first tantalum circuit layer made of tantalum (Ta), a first tungsten circuit layer made of tungsten (W), a first copper-aluminum (AlCu) alloy circuit layer, a first copper-aluminum-silicon (AlSiCu) alloy circuit layer, a first tantalum nitride (TaN) circuit layer or a first titanium nitride (TiN) circuit layer. In addition, depending on different requirements, the second conductive circuit layer 3B can be configured as a second gold circuit layer made of gold (Au), a second silver circuit layer made of silver (Ag), a second copper circuit layer made of copper (Cu), a second aluminum circuit layer made of aluminum (Al), a second nickel circuit layer made of nickel (Ni), a second titanium circuit layer made of titanium (Ti), a second platinum circuit layer made of platinum (Pt), a second palladium circuit layer made of palladium (Pd), a second tantalum circuit layer made of tantalum (Ta), a second tungsten circuit layer made of tungsten (W), a second copper-aluminum (AlCu) alloy circuit layer, a second copper-aluminum-silicon (AlSiCu) alloy circuit layer, a second tantalum nitride (TaN) circuit layer or a second titanium nitride (TiN) circuit layer. It should be noted that, according to different requirements, the thickness (or the height) of the first conductive circuit layer 3A can be between 200 Å and 4000 Å (such as any positive integer between 200 Å and 4000 Å), and the thickness (or the height) of the second conductive circuit layer 3B can be between 200 Å and 4000 Å (such as any positive integer between 200 Å and 4000 Å). However, the aforementioned details are disclosed for exemplary purposes only, and are not meant to limit the scope of the present disclosure.

For example, referring to FIG. 6 and FIG. 7, depending on different requirements, each of the first conductive through layers 4A can be configured as a first gold via made of gold (Au), a first silver via made of silver (Ag), a first copper via made of copper (Cu), a first aluminum via made of aluminum (Al), a first nickel via made of nickel (Ni), a first titanium via made of titanium (Ti), a first titanium via made of titanium (Ti), a platinum via made of platinum (Pt), a first palladium via made of palladium (Pd), a first tantalum via made of tantalum (Ta), a first tungsten via made of tungsten (W), a first copper-aluminum (AlCu) alloy a through hole, a first copper-aluminum-silicon (AlSiCu) alloy through hole, a first tantalum nitride (TaN) through hole or a first titanium nitride (TiN) through hole. In addition, each of the second conductive through layers 4B can be configured as a second gold via made of gold (Au), a second silver via made of silver (Ag), a second copper via made of copper (Cu), a second aluminum via made of aluminum (Al), a second nickel via made of nickel (Ni), a second titanium via made of titanium (Ti), a second titanium via made of titanium (Ti), a platinum via made of platinum (Pt), a second palladium via made of palladium (Pd), a second tantalum via made of tantalum (Ta), a second tungsten via made of tungsten (W), a second copper-aluminum (AlCu) alloy a through hole, a second copper-aluminum-silicon (AlSiCu) alloy through hole, a second tantalum nitride (TaN) through hole or a second titanium nitride (TiN) through hole. It should be noted that, according to different requirements, the thickness (or the height) of each of the first conductive through layers 4A can be between 1500 Å and 2500 Å (such as any positive integer between 1500 Å and 2500 Å), and the width of each of the first conductive through layers 4A can be between 50 nm and 200 nm (such as any positive integer between 50 nm and 200 nm). In addition, the thickness (or the height) of each of the second conductive through layers 4B can be between 1500 Å and 2500 Å (such as any positive integer between 1500 Å and 2500 Å), and the width of each of the second conductive through layers 4B can be between 50 nm and 200 nm (such as any positive integer between 50 nm and 200 nm). However, the aforementioned details are disclosed for exemplary purposes only, and are not meant to limit the scope of the present disclosure.

For example, referring to FIG. 8 and FIG. 9, depending on different requirements, each of the first exposed electrodes 5A can be configured as a working electrode, and each of the first exposed electrodes 5A can be configured as a first gold electrode made of gold (Au), a first silver electrode made of silver (Ag), a first copper electrode made of copper (Cu), a first aluminum electrode made of aluminum (Al), a first nickel electrode made of nickel (Ni), a first titanium electrode made of titanium (Ti), a first platinum electrode made of platinum (Pt), a first palladium electrode made of palladium (Pd), a first tantalum electrode made of tantalum (Ta), a first tungsten electrode made of tungsten (W), a first copper-aluminum (AlCu) alloy electrode, a first copper-aluminum-silicon (AlSiCu) alloy electrode, a first tantalum nitride (TaN) electrode or a first titanium nitride (TiN) electrode. In addition, each of the second exposed electrodes 5B can be configured as a counter electrode, and each of the second exposed electrodes 5B can be configured as a second gold electrode made of gold (Au), a second silver electrode made of silver (Ag), a second copper electrode made of copper (Cu), a second aluminum electrode made of aluminum (Al), a second nickel electrode made of nickel (Ni), a second titanium electrode made of titanium (Ti), a second platinum electrode made of platinum (Pt), a second palladium electrode made of palladium (Pd), a second tantalum electrode made of tantalum (Ta), a second tungsten electrode made of tungsten (W), a second copper-aluminum (AlCu) alloy electrode, a second copper-aluminum-silicon (AlSiCu) alloy electrode, a second tantalum nitride (TaN) electrode or a second titanium nitride (TiN) electrode. It should be noted that, according to different requirements, the thickness (or the height) of each of the first exposed electrodes 5A can be between 500 Å and 50000 Å (such as any positive integer between 500 Å and 50000 Å), the width of each of the first exposed electrodes 5A can be between 100 nm and 5000 nm (such as any positive integer between 100 nm and 5000 nm), and the distance between two adjacent ones of the plurality of first exposed electrodes 5A can be between 1000 nm and 10000 nm (such as any positive integer between 1000 nm and 10000 nm). In addition, the thickness (or the height) of each of the second exposed electrodes 5B can be between 500 Å and 50000 Å (such as any positive integer between 500 Å and 50000 Å), and the width of each of the second exposed electrodes 5B can be between 3000 nm and 5000 nm (such as any positive integer between 3000 nm and 5000 nm). However, the aforementioned details are disclosed for exemplary purposes only, and are not meant to limit the scope of the present disclosure.

It should be noted that, for example, in the process (the step S102) of forming the first conductive circuit layer 3A and the second conductive circuit layer 3B, the biosensor manufacturing method further includes: forming a third conductive circuit layer 3C (as shown in FIG. 2) that is disposed on the first insulating layer 2A. Moreover, in the process (the step S106) of forming the first conductive through layers 4A and the second conductive through layers 4B, the biosensor manufacturing method further includes: forming a plurality of third conductive through layers 4C (as shown in FIG. 6) to pass through the second insulating layer 2B and to be electrically connected to the third conductive circuit layer 3C. In addition, in the process (the step S108) of forming the first exposed electrodes 5A and the second exposed electrodes 5B, the biosensor manufacturing method further includes: forming a third exposed electrode 5C (as shown in FIG. 8) to be disposed on the second insulating layer 2B and electrically connected to the third conductive through layers 4C, respectively. That is to say, referring to FIG. 2, FIG. 6 and FIG. 8, the biosensor S further includes a third conductive circuit layer 3C, a plurality of third conductive through layers 4C and a third exposed electrode 5C, the third conductive through layers 4C are electrically connected to the third conductive circuit layer 3C and the third exposed electrode 5C, and the third exposed electrode 5C can be configured as a reference electrode. It should be noted that the third conductive circuit layer 3C can be made of the same material as the first conductive circuit layer 3A or the second conductive circuit layer 3B, the third conductive through layer 4C can be made of the same material as the first conductive through layer 4A or the second conductive through layer 4B, and the third exposed electrode 5C can be made of the same material as the first exposed electrode 5A or the second exposed electrode 5B. However, the aforementioned details are disclosed for exemplary purposes only, and are not meant to limit the scope of the present disclosure.

### Second Embodiment

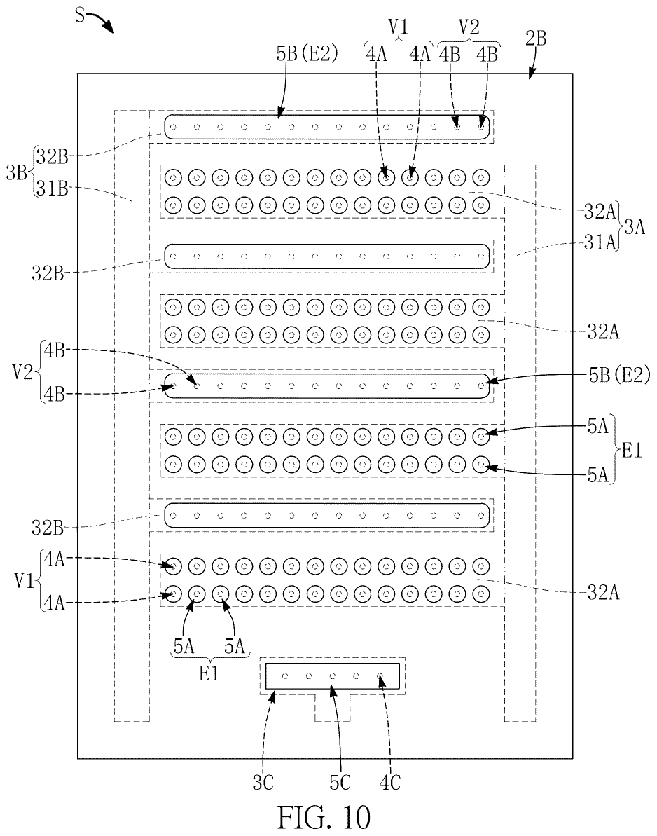

Referring to FIG. 10, a second embodiment of the present disclosure provides a biosensor S. Comparing FIG. 10 with FIG. 8, the main difference between the second embodiment and the first embodiment is as follows: in the second embodiment, a second exposed electrode 5B (or a continuous second exposed electrode) of each second electrode array E2 can be disposed above the corresponding second circuit portion 32B of the second conductive circuit layer 3B and be electrically connected to the second conductive through layers 4B of a corresponding one of the second via arrays V2, respectively. That is to say, according to different requirements, each second electrode array E2 may include a plurality of second exposed electrodes 5B (as shown in the first embodiment) or only one second exposed electrode 5B (as shown in the second embodiment). However, the aforementioned details are disclosed for exemplary purposes only, and are not meant to limit the scope of the present disclosure.

### Beneficial Effects of the Embodiments

In conclusion, in the biosensor S provided by the present disclosure, by virtue of “the first conductive through layers 4A passing through the second insulating layer 2B to be electrically connected to the first conductive circuit layer 3A,” “the second conductive through layers 4B passing through the second insulating layer 2B to be electrically connected to the second conductive circuit layer 3B,” “the first exposed electrodes 5A being disposed on the second insulating layer 2B, and each of the first exposed electrodes 5A being electrically connected to a corresponding one of the first conductive through layers 4A” and “the second exposed electrodes 5B being disposed on the second insulating layer 2B, and each of the second exposed electrodes 5B being electrically connected to a corresponding one of the second conductive through layers 4B,” the first exposed electrode 5A can be electrically connected to the first conductive circuit layer 3A through the corresponding first conductive through layer 4A and be separate from the first conductive circuit layer 3A by a first predetermined distance, and the second exposed electrode 5B can be electrically connected to the second conductive circuit layer 3B through the corresponding second conductive through layer 4B and be separate from the second conductive circuit layer 3B by a second predetermined distance.

Furthermore, in the biosensor manufacturing method provided by the present disclosure, by virtue of “forming a plurality of first conductive through layers 4A and a plurality of second conductive through layers 4B, the first conductive through layers 4A passing through the second insulating layer 2B to be electrically connected to the first conductive circuit layer 3A, and the second conductive through layers 4B passing through the second insulating layer 2B to be electrically connected to the second conductive circuit layer 3B” and “forming a plurality of first exposed electrodes 5A and a plurality of second exposed electrodes 5B, the first exposed electrodes 5A being disposed on the second insulating layer 2B and electrically connected to the first conductive through layers 4A, respectively, and the second exposed electrodes 5B being disposed on the second insulating layer 2B and electrically connected to the second conductive through layers 4B, respectively,” the first exposed electrode 5A can be electrically connected to the first conductive circuit layer 3A through the corresponding first conductive through layer 4A and be separate from the first conductive circuit layer 3A by a first predetermined distance, and the second exposed electrode 5B can be electrically connected to the second conductive circuit layer 3B through the corresponding second conductive through layer 4B and be separate from the second conductive circuit layer 3B by a second predetermined distance.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A biosensor, comprising:
a carrier substrate;
a first insulating layer disposed on the carrier substrate;
a first conductive circuit layer disposed on the first insulating layer;
a second conductive circuit layer disposed on the first insulating layer;
a second insulating layer disposed on the first insulating layer for partially covering the first conductive circuit layer and the second conductive circuit layer;
a plurality of first conductive through layers passing through the second insulating layer to be electrically connected to the first conductive circuit layer;
a plurality of second conductive through layers passing through the second insulating layer to be electrically connected to the second conductive circuit layer;
a plurality of first exposed electrodes disposed on the second insulating layer, wherein each of the plurality of first exposed electrodes is electrically connected to a corresponding one of the plurality of first conductive through layers; and
a plurality of second exposed electrodes disposed on the second insulating layer, wherein each of the plurality of second exposed electrodes is electrically connected to a corresponding one of the plurality of second conductive through layers;
wherein the first conductive circuit layer and the second conductive circuit layer are adjacent to each other and separate from each other, and the first conductive circuit layer and the second conductive circuit layer cooperate with each other to form an interdigitated electrode structure; and
wherein the first conductive circuit layer includes a first extension portion and a plurality of first circuit portions extending from the first extension portion, the second conductive circuit layer includes a second extension portion and a plurality of second circuit portions extending from the second extension portion, and the plurality of first circuit portions of the first conductive circuit layer and the plurality of second circuit portions of the second conductive circuit layer are alternately arranged.

2. The biosensor according to claim 1,
wherein the plurality of first circuit portions of the first conductive circuit layer are parallel to each other, and the plurality of first circuit portions extend vertically or obliquely from the first extension portion;
wherein the plurality of second circuit portions of the second conductive circuit layer are parallel to each other, and the plurality of second circuit portions extend vertically or obliquely from the second extension portion;
wherein the first extension portion of the first conductive circuit layer and the second extension portion of the second conductive circuit layer are parallel to each other, and the plurality of first circuit portions of the first conductive circuit layer and the plurality of second circuit portions of the second conductive circuit layer are parallel to each other;
wherein the first conductive circuit layer and the second conductive circuit layer cooperate with each other to form a continuous meandering gap between the first conductive circuit layer and the second conductive circuit layer;
wherein a first end and a second end of each of the plurality of first conductive through layers are in electrical contact with the first conductive circuit layer and a corresponding one of the plurality of first exposed electrodes, respectively;
wherein a first end and a second end of each of the plurality of second conductive through layers are in electrical contact with the second conductive circuit layer and a corresponding one of the plurality of second exposed electrodes, respectively;
wherein the plurality of first conductive through layers are divided into a plurality of first via arrays, and the first conductive through layers of each of the plurality of first via arrays are electrically connected to a corresponding one of the plurality of first circuit portions of the first conductive circuit layer;
wherein the plurality of first exposed electrodes are divided into a plurality of first electrode arrays, and the first exposed electrodes of each of the plurality of first electrode arrays are disposed above a corresponding one of the first circuit portions of the first conductive circuit layer and electrically connected to the plurality of first conductive through layers of a corresponding one of the plurality of first via arrays, respectively;
wherein the plurality of second conductive through layers are divided into a plurality of second via arrays, and the second conductive through layers of each of the plurality of second via arrays are electrically connected to a corresponding one of the plurality of second circuit portions of the second conductive circuit layer;
wherein the plurality of second exposed electrodes are divided into a plurality of second electrode arrays, and the second exposed electrodes of each of the plurality of second electrode arrays are disposed above a corresponding one of the second circuit portions of the second conductive circuit layer and electrically connected to the plurality of second conductive through layers of a corresponding one of the plurality of second via arrays, respectively;
wherein each of the plurality of first exposed electrodes is configured to present a columnar shape, and each of the plurality of second exposed electrodes is configured to present a strip shape;
wherein each of the plurality of first exposed electrodes is electrically connected to one or more corresponding ones of the plurality of first conductive through layers, and each of the plurality of second exposed electrodes is electrically connected to one or more corresponding ones of the plurality of second conductive through layers;
wherein a thickness of the first insulating layer is between 3500 Å and 4500 Å, and a thickness of the second insulating layer is between 4000 Å and 15000 Å;
wherein a thickness of the first conductive circuit layer is between 200 Å and 4000 Å, a thickness of the second conductive circuit layer is between 200 Å and 4000 Å, and a width of the continuous meandering gap is between 2000 nm and 6000 nm;
wherein a thickness of each of the plurality of first conductive through layers is between 1500 Å and 2500 Å, and a width of each of the plurality of first conductive through layers is between 50 nm and 200 nm;
wherein a thickness of each of the plurality of second conductive through layers is between 1500 Å and 2500 Å, and a width of each of the plurality of second conductive through layers is between 50 nm and 200 nm;
wherein a thickness of each of the plurality of first exposed electrodes is between 500 Å and 50000 Å, a width of each of the plurality of first exposed electrodes is between 100 nm and 5000 nm, and a distance between two adjacent ones of the plurality of first exposed electrodes is between 1000 nm and 10000 nm; and
wherein a thickness of each of the plurality of second exposed electrodes is between 500 Å and 50000 Å, and a width of each of the plurality of second exposed electrodes is between 3000 nm and 5000 nm.

3. The biosensor according to claim 1,
wherein the carrier substrate is configured as a silicon wafer substrate, a gallium nitride substrate, a silicon carbide substrate, a silicon germanium substrate, a sapphire substrate or a glass substrate;
wherein the first insulating layer is configured as a first oxide layer, a first nitride layer or a first oxynitride layer;
wherein the second insulating layer is configured as a second oxide layer, a second nitride layer or a first oxynitride layer;
wherein the first conductive circuit layer is configured as a first gold circuit layer made of gold, a first silver circuit layer made of silver, a first copper circuit layer made of copper, a first aluminum circuit layer made of aluminum, a first nickel circuit layer made of nickel, a first titanium circuit layer made of titanium, a first platinum circuit layer made of platinum, a first palladium circuit layer made of palladium, a first tantalum circuit layer made of tantalum, a first tungsten circuit layer made of tungsten, a first copper-aluminum alloy circuit layer, a first copper-aluminum-silicon alloy circuit layer, a first tantalum nitride circuit layer or a first titanium nitride circuit layer;
wherein the second conductive circuit layer is configured as a second gold circuit layer made of gold, a second silver circuit layer made of silver, a second copper circuit layer made of copper, a second aluminum circuit layer made of aluminum, a second nickel circuit layer made of nickel, a second titanium circuit layer made of titanium, a second platinum circuit layer made of platinum, a second palladium circuit layer made of palladium, a second tantalum circuit layer made of tantalum, a second tungsten circuit layer made of tungsten, a second copper-aluminum alloy circuit layer, a second copper-aluminum-silicon alloy circuit layer, a second tantalum nitride circuit layer or a second titanium nitride circuit layer;
wherein each of the plurality of first conductive through layers is configured as a first gold via made of gold, a first silver via made of silver, a first copper via made of copper, a first aluminum via made of aluminum, a first nickel via made of nickel, a first titanium via made of titanium, a first titanium via made of titanium, a platinum via made of platinum, a first palladium via made of palladium, a first tantalum via made of tantalum, a first tungsten via made of tungsten, a first copper-aluminum alloy a through hole, a first copper-aluminum-silicon alloy through hole, a first tantalum nitride through hole or a first titanium nitride through hole;
wherein each of the plurality of second conductive through layers is configured as a second gold via made of gold, a second silver via made of silver, a second copper via made of copper, a second aluminum via made of aluminum, a second nickel via made of nickel, a second titanium via made of titanium, a second titanium via made of titanium, a platinum via made of platinum, a second palladium via made of palladium, a second tantalum via made of tantalum, a second tungsten via made of tungsten, a second copper-aluminum alloy a through hole, a second copper-aluminum-silicon alloy through hole, a second tantalum nitride through hole or a second titanium nitride through hole;
wherein each of the plurality of first exposed electrodes is configured as a working electrode, and each of the plurality of first exposed electrodes is configured as a first gold electrode made of gold, a first silver electrode made of silver, a first copper electrode made of copper, a first aluminum electrode made of aluminum, a first nickel electrode made of nickel, a first titanium electrode made of titanium, a first platinum electrode made of platinum, a first palladium electrode made of palladium, a first tantalum electrode made of tantalum, a first tungsten electrode made of tungsten, a first copper-aluminum alloy electrode, a first copper-aluminum-silicon alloy electrode, a first tantalum nitride electrode or a first titanium nitride electrode;
wherein each of the plurality of second exposed electrodes is configured as a counter electrode, and each of the plurality of second exposed electrodes is configured as a second gold electrode made of gold, a second silver electrode made of silver, a second copper electrode made of copper, a second aluminum electrode made of aluminum, a second nickel electrode made of nickel, a second titanium electrode made of titanium, a second platinum electrode made of platinum, a second palladium electrode made of palladium, a second tantalum electrode made of tantalum, a second tungsten electrode made of tungsten, a second copper-aluminum alloy electrode, a second copper-aluminum-silicon alloy electrode, a second tantalum nitride electrode or a second titanium nitride electrode; and
wherein the biosensor further includes a third conductive circuit layer, a plurality of third conductive through layers and a third exposed electrode, the plurality of third conductive through layers are electrically connected to the third conductive circuit layer and the third exposed electrode, and the third exposed electrode is configured as a reference electrode.

4. A biosensor, comprising:
a carrier substrate;
a first insulating layer disposed on the carrier substrate;
a first conductive circuit layer disposed on the first insulating layer;
a second conductive circuit layer disposed on the first insulating layer;
a second insulating layer disposed on the first insulating layer for partially covering the first conductive circuit layer and the second conductive circuit layer;
a plurality of first conductive through layers passing through the second insulating layer to be electrically connected to the first conductive circuit layer;
a plurality of second conductive through layers passing through the second insulating layer to be electrically connected to the second conductive circuit layer;
a plurality of first exposed electrodes disposed on the second insulating layer, wherein each of the plurality of first exposed electrodes is electrically connected to a corresponding one of the plurality of first conductive through layers; and
a plurality of second exposed electrodes disposed on the second insulating layer, wherein each of the plurality of second exposed electrodes is electrically connected to a corresponding one of the plurality of second conductive through layers.

5. The biosensor according to claim 4,
wherein the first conductive circuit layer and the second conductive circuit layer are adjacent to each other and separate from each other, and the first conductive circuit layer and the second conductive circuit layer cooperate with each other to form an interdigitated electrode structure; and
wherein the first conductive circuit layer and the second conductive circuit layer cooperate with each other to form a continuous meandering gap between the first conductive circuit layer and the second conductive circuit layer.

6. The biosensor according to claim 4,
wherein each of the plurality of first exposed electrodes is configured as a working electrode, and each of the plurality of second exposed electrodes is configured as a counter electrode; and
wherein the biosensor further includes a third conductive circuit layer, a plurality of third conductive through layers and a third exposed electrode, the plurality of third conductive through layers are electrically connected to the third conductive circuit layer and the third exposed electrode, and the third exposed electrode is configured as a reference electrode.

7. A biosensor manufacturing method, comprising:
forming a first insulating layer disposed on a carrier substrate;
forming a first conductive circuit layer and a second conductive circuit layer on the first insulating layer;
forming a second insulating layer on the first insulating layer for partially covering the first conductive circuit layer and the second conductive circuit layer;
forming a plurality of first conductive through layers and a plurality of second conductive through layers, wherein the plurality of first conductive through layers pass through the second insulating layer to be electrically connected to the first conductive circuit layer, and the plurality of second conductive through layers pass through the second insulating layer to be electrically connected to the second conductive circuit layer; and
forming a plurality of first exposed electrodes and a plurality of second exposed electrodes, wherein the plurality of first exposed electrodes are disposed on the second insulating layer and electrically connected to the plurality of first conductive through layers, respectively, and the plurality of second exposed electrodes are disposed on the second insulating layer and electrically connected to the plurality of second conductive through layers, respectively.

8. The biosensor manufacturing method according to claim 7,
wherein, after the process of forming the plurality of first exposed electrodes and the plurality of second exposed electrodes, the biosensor manufacturing method further comprises: performing a cleaning process to clean a biosensor manufactured by the biosensor manufacturing method;
wherein, after the process of forming the plurality of first exposed electrodes and the plurality of second exposed electrodes, the biosensor manufacturing method further comprises: performing a leveling process to improve the surface flatness of each of the plurality of first exposed electrodes and the surface flatness of each of the plurality of second exposed electrodes;
wherein, in the process of forming the first conductive circuit layer and the second conductive circuit layer, the biosensor manufacturing method further comprises: forming a third conductive circuit layer that is disposed on the first insulating layer;
wherein, in the process of forming the plurality of first conductive through layers and the plurality of second conductive through layers, the biosensor manufacturing method further comprises: forming a plurality of third conductive through layers to pass through the second insulating layer and to be electrically connected to the third conductive circuit layer; and
wherein, in the process of forming the plurality of first exposed electrodes and the plurality of second exposed electrodes, the biosensor manufacturing method further comprises: forming a third exposed electrode to be disposed on the second insulating layer and electrically connected to the plurality of third conductive through layers, respectively.

9. The biosensor manufacturing method according to claim 7,
wherein the first conductive circuit layer and the second conductive circuit layer are adjacent to each other and separate from each other, and the first conductive circuit layer and the second conductive circuit layer cooperate with each other to form an interdigitated electrode structure;
wherein the first conductive circuit layer includes a first extension portion and a plurality of first circuit portions extending from the first extension portion, the second conductive circuit layer includes a second extension portion and a plurality of second circuit portions extending from the second extension portion, and the plurality of first circuit portions of the first conductive circuit layer and the plurality of second circuit portions of the second conductive circuit layer are alternately arranged;
wherein the plurality of first circuit portions of the first conductive circuit layer are parallel to each other, and the plurality of first circuit portions extend vertically or obliquely from the first extension portion;
wherein the plurality of second circuit portions of the second conductive circuit layer are parallel to each other, and the plurality of second circuit portions extend vertically or obliquely from the second extension portion;
wherein the first extension portion of the first conductive circuit layer and the second extension portion of the second conductive circuit layer are parallel to each other, and the plurality of first circuit portions of the first conductive circuit layer and the plurality of second circuit portions of the second conductive circuit layer are parallel to each other;
wherein the first conductive circuit layer and the second conductive circuit layer cooperate with each other to form a continuous meandering gap between the first conductive circuit layer and the second conductive circuit layer;
wherein a first end and a second end of each of the plurality of first conductive through layers are in electrical contact with the first conductive circuit layer and a corresponding one of the plurality of first exposed electrodes, respectively;
wherein a first end and a second end of each of the plurality of second conductive through layers are in electrical contact with the second conductive circuit layer and a corresponding one of the plurality of second exposed electrodes, respectively;
wherein the plurality of first conductive through layers are divided into a plurality of first via arrays, and the first conductive through layers of each of the plurality of first via arrays are electrically connected to a corresponding one of the plurality of first circuit portions of the first conductive circuit layer;
wherein the plurality of first exposed electrodes are divided into a plurality of first electrode arrays, and the first exposed electrodes of each of the plurality of first electrode arrays are disposed above a corresponding one of the first circuit portions of the first conductive circuit layer and electrically connected to the plurality of first conductive through layers of a corresponding one of the plurality of first via arrays, respectively;
wherein the plurality of second conductive through layers are divided into a plurality of second via arrays, and the second conductive through layers of each of the plurality of second via arrays are electrically connected to a corresponding one of the plurality of second circuit portions of the second conductive circuit layer; and
wherein the plurality of second exposed electrodes are divided into a plurality of second electrode arrays, and the second exposed electrodes of each of the plurality of second electrode arrays are disposed above a corresponding one of the second circuit portions of the second conductive circuit layer and electrically connected to the plurality of second conductive through layers of a corresponding one of the plurality of second via arrays, respectively.

10. The biosensor manufacturing method according to claim 7,
wherein each of the plurality of first exposed electrodes is configured as a working electrode, and each of the plurality of second exposed electrodes is configured as a counter electrode; and
wherein the biosensor further includes a third conductive circuit layer, a plurality of third conductive through layers and a third exposed electrode, the plurality of third conductive through layers are electrically connected to the third conductive circuit layer and the third exposed electrode, and the third exposed electrode is configured as a reference electrode.
